(19) United States
(12) Patent Application Publication
(10) Pub. No.: US 2003/0032941 A1
(43) Pub. Date: Feb. 13, 2003

(54) Convertible delivery systems for medical devices

(76) Inventors: William J. Boyle, Fallbrook, CA (US); Benjamin C. Huter, Murrieta, CA (US); John E. Papp, Temecula, CA (US); Jack Sahakian, Escondido, CA (US); Richard S. Stack, Chapel Hills, NC (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/928,464

(22) Filed: Aug. 13, 2001

(51) Int. Cl. A61M 25/16

(52) U.S. Cl. 604/533

(57) ABSTRACT

A delivery system for a medical device or other devices to be deployed within a biological body including a sheath having a longitudinal joint. The joint can be either resealable or non-resealable. The joint remains intact during delivery of the medical device, thereby facilitating accurate delivery of the medical device. During removal of the sheath, the joint is separated, thereby permitting the sheath to be peeled from the medical device while maintaining the position of the device. The delivery system allows a single operator to deploy the medical device and remove the sheath.

### BACKGROUND OF THE INVENTION

The present invention relates generally to delivery systems for medical devices and, more particularly, to systems which can be used when an interventional procedure is being performed in a stenosed or occluded region of a body vessel.

Numerous procedures have been developed for treating occluded blood vessels to allow blood to flow without obstruction. Such procedures usually involve the percutaneous introduction of an interventional device into the lumen of the artery, usually through a catheter. One widely known and medically accepted procedure is balloon angioplasty in which an inflatable balloon is introduced within the stenosed region of the blood vessel to dilate the occluded vessel. The balloon catheter is initially inserted into the patient's arterial system and is advanced and manipulated into the area of stenosis in the artery. The balloon is inflated to compress plaque or other material at the treatment site and press the vessel wall radially outward to increase the diameter of the blood vessel to thereby result in increasing blood flow. The balloon is then deflated to a small profile so that the balloon catheter can be withdrawn from the patient's vasculature. As should be appreciated by those skilled in the art, while the above-described procedure is typical, it is not the only method used in angioplasty.

Another treatment procedure is laser angioplasty that utilizes a laser to ablate the stenosis by super heating and vaporizing the deposited plaque. Atherectomy is yet another method of treating a stenosed blood vessel in which cutting blades are rotated to shave the deposited plaque from the arterial wall. A vacuum catheter is usually used to capture the shaved plaque or thrombus from the blood stream during this procedure.

In the procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician may implant an intravascular prosthesis, commonly known as a stent, for maintaining vascular patency inside the artery across the lesion. The stent can be crimped onto the balloon portion of the catheter and transported in its delivery diameter through the patient's vasculature. At the deployment site, the stent is expanded to a larger diameter, often by inflating the balloon portion of the catheter.

The above minimally invasive interventional procedures, when successful, avoid the necessity of major surgical operations. However, there is one common problem that can become associated with all of these procedures, namely, the potential release of embolic debris into the bloodstream that can occlude distal vasculature and cause significant health problems to the patient. For example, during deployment of a stent, it is possible that the metal struts of the stent can cut into the stenosis and shear off pieces of plaque which become embolic debris that can travel downstream and lodge somewhere in the patient's vascular system. Pieces of plaque material also can sometimes dislodge from the stenosis during a balloon angioplasty procedure and become released into the bloodstream. Additionally, while complete vaporization of plaque is the intended goal during laser angioplasty, sometimes particles are not fully vaporized and thus enter the bloodstream. Likewise, not all of the emboli created during an atherectomy procedure may be drawn into the vacuum catheter and, as a result, enter the bloodstream.

When any of the above-described procedures are performed in the carotid arteries, the release of emboli into the circulatory system can be extremely dangerous and sometimes fatal to the patient. Debris that is carried by the bloodstream to distal vessels of the brain can cause these cerebral vessels to occlude, resulting in a stroke, and in some cases, death. Therefore, although cerebral percutaneous transluminal angioplasty has been performed in the past, the number of procedures performed has been limited due to the justifiable fear of causing an embolic stroke should embolic debris enter the bloodstream and block vital downstream blood passages.

Medical devices have been developed to attempt to deal with the problem created when debris or fragments enter the circulatory system following vessel treatment utilizing any of the above-identified procedures. One approach that has been attempted is the cutting of any debris into minute sizes which pose little chance of becoming occluded in major vessels within the patient's vasculature. However, it is often difficult to control the size of the fragments that are formed, and the potential risk of vessel occlusion still exists, making such a procedure in the carotid arteries a high-risk proposition.

Other techniques include the use of catheters with a vacuum source that provides temporary suction to remove embolic debris from the bloodstream. However, as mentioned above, there can be complications associated with such systems if the vacuum catheter does not remove all of the embolic material from the bloodstream. Also, a powerful suction could cause trauma to the patient's vasculature. Still other techniques that have had some success include the placement of a filter or trap downstream from the treatment site to capture embolic debris before it reaches the smaller blood vessels downstream. The placement of a filter in the patient's vasculature during treatment of the vascular lesion can reduce the presence of the embolic debris in the bloodstream. Such embolic filters are usually delivered in a collapsed position through the patient's vasculature and then expanded to trap the embolic debris. Some of these embolic filters are self expanding and utilize a restraining sheath that maintains the expandable filter in a collapsed position until it is ready to be expanded within the patient's vasculature. The physician can retract the proximal end of the restraining sheath to expose the expandable filter, causing the filter to expand at the desired location. Once the procedure is completed, the filter can be collapsed, and the filter (with the trapped embolic debris) can then be removed from the vessel.

Some prior art expandable vessel filters are attached to a distal end of a guide wire or guide wire-like tubing that allows the filtering device to be placed in the patient's vasculature as the guide wire is steered by the physician. Once the guide wire is in the proper position within the vasculature, the embolic filter can be deployed to capture embolic debris. Some embolic filter devices that utilize a guide wire for positioning also utilize a restraining sheath to maintain the expandable filter in a collapsed configuration. Once the proximal end of the restraining sheath is retracted by the physician, the expandable filter will move into its fully expanded position within the patient's vasculature. The restraining sheath can then be removed from the guide wire allowing the guide wire to be used by the physician to deliver interventional devices, such as a balloon angioplasty dilatation catheter or a stent delivery catheter, into the area of treatment. After the interventional procedure is completed a recovery sheath can be delivered over the guide wire using over-the-wire techniques to collapse the expanded filter for removal from the patient's vasculature.

Some prior art catheters for delivering expandable filters or interventional devices utilize a delivery sheath to first deliver the guide wire, filter or interventional device within the corporeal vessel. When the guide wire is in position, the delivery sheath is removed and the expandable filter or interventional device is deployed. In a typical over-the-wire delivery platform, the guide wire may be more than twice the length of the delivery sheath with more than half the length external to the patient during the delivery of the expandable filter. This extra length is needed when the delivery sheath is removed from the patient since the guide wire must usually be held in place. Therefore, the portion of the guide wire external the patient must be longer than the delivery sheath to allow the operator to grasp a portion of the guide wire during all stages of delivery sheath removal. Due to the length of the guide wire, it may sometimes be necessary to have a second person assist the operator when removing the delivery sheath to prevent the guide wire from shifting within the vessel.

Other delivery catheters may utilize a rapid exchange or monorail delivery platform to deliver the guide wire. The typical rapid exchange delivery platform may include a 20 to 30 cm long lumen for the guide wire at the distal portion of the delivery sheath, with the remainder of the guide wire being located outside the catheter sheath. The portion of the guide wire outside the lumen of the delivery sheath may be about 100 cm long. With a rapid exchange delivery platform, the length of the portion of the guide wire external the patient can be much shorter, permitting the sheath to be removed by only one person. However, when a rapid exchange platform is used, the delivery sheath may not have as much axial rigidity or stiffness as when a full sized sheath is used or when an over-the-wire procedure is employed. This may lead to excess play or “splay” between the guide wire and the delivery sheath during delivery.

What has been needed are delivery platform systems for guide wire based filtering devices or other medical devices which combine the benefits of delivering the guide wire via an over-the-wire platform with the benefits of removing the delivery sheath via a rapid exchange or monorail platform. These systems should provide the benefit of smooth device delivery common to standard over-the-wire platforms along with the benefit of being removable by a single operator. The inventions disclosed herein satisfy these and other needs.

### SUMMARY OF THE INVENTION

Briefly, and in general terms, the present invention is directed to a delivery system for a medical device for use in a biological body. More particularly, the delivery system is directed to the delivery of filtering devices or other interventional devices via an over-the-wire platform, yet allows the devices to be recovered via a rapid exchange platform.

In one aspect, the invention relates to a delivery sheath for an embolic protection device. The sheath includes an elongate tube with a proximal end and a distal end. The sheath also includes a longitudinal joint that can be either resealable, or include a slit that is non-resealable.

In one embodiment, the resealable longitudinal joint includes a first side and a second side. The first side has a protrusion with a neck that leads to a head that is larger than the neck. The second side has an opening that leads to a cavity. The opening is smaller than the head of the first side and at least as large as the neck of the first side. The cavity is within a range of slightly smaller than the head of the first side to larger than the head of the first side. The longitudinal joint can extend either an entire length of the sheath or extend from a proximal end of the sheath to a position proximal to the distal end of the sheath, such as between 20 to 40 centimeters proximal to the distal end of the sheath.

The sheath can include a single or a plurality of longitudinally extending lumens. The sheath may also include an elongate tube having at least a first lumen and a second lumen with a reduced cross sectional area positioned adjacent the first lumen. The reduced cross sectional area defines a groove penetrating an outer surface of the sheath.

The sheath is contemplated to be part of a delivery system for delivering embolic protection devices. The delivery system can further include a guide wire that extends through a length of the at least one lumen of the sheath in a coaxial fashion as well as a device that accomplishes splitting the longitudinal joint. A device couples a distal portion of a handle to a proximal portion of the sheath.

The device for splitting the longitudinal joint includes a ring dimensioned to receive a sheath and having an internal bore configured with a plow, the height of which is sufficient to extend into the lumen of the sheath. A distal portion of the plow is configured to enter and split the longitudinal joint during relative longitudinal movement between the ring and the sheath. Alternatively, the ring may have a blade including a cutting edge for cutting the sheath during relative longitudinal movement between the ring and the sheath. A guide mandrel can be coupled to the blade to facilitate control of the longitudinal motion of the blade.

A method using the delivery system to deliver and deploy an embolic protection or other medical device within a biological body vessel includes introducing the device with the delivery system into the body vessel, advancing the device to the desired location within the body vessel, deploying the device at the desired location within the body vessel, and removing the sheath from the guide wire.

Deploying the embolic protection or other medical device includes holding the device in a relatively steady position while extracting the sheath proximally to thereby expose the device. The sheath may be removed form the guide wire by detaching the handle and the device coupling the handle to the sheath from the proximal portion of the sheath. While holding the device and the sheath in a relatively steady position, the sheath removal ring is advanced distally toward the entry point of the delivery system into the biological body, thereby splitting the longitudinal joint at the proximal portion of the sheath. The portion of the sheath proximal to the sheath removal ring is then pulled proximally while holding the sheath removal ring and the device in a relatively steady position until the distal end of the longitudinal joint is external to the biological body. The sheath removal ring is then removed while holding the device in a relatively steady position. The remainder of the sheath may then be removed from the vasculature.

Alternate embodiments of the sheath include the longitudinal joint including a longitudinal slit that is configured such that the guide wire does not inadvertently dislodge itself from the sheath through the slit. One embodiment accomplishes this by configuring the slit with a curved profile, such as an s-shape. Another embodiment configures the sheath with varying thicknesses about the lumen such that the area adjacent the slit has a smaller cross section than the surrounding areas with the thicker areas providing structural support to the thinner areas. Another embodiment includes a sheath having at least a first lumen and a second lumen with a groove running longitudinally along the side of the sheath and throughout the length of the sheath. Another configuration of this embodiment includes a longitudinal slit running through the groove from the proximal end of the sheath to a location between 20 to 40 cm proximal to the distal end of the sheath. With these embodiments, removing the sheath from the guide wire includes peeling the sheath from the guide wire by causing the guide wire to either pull through the slit or to tear through the groove.

These and other aspects and advantages of the invention will become apparent from the following detailed description and the accompanying drawings, which illustrate by way of example the features of the invention.

### BRIEF DESCRIPTION OF THE DRAWINGS

FIG. 1a is a view depicting a medical device deployed within a human body via an over-the-wire method.

FIG. 1b is a view depicting a medical device deployed within a human body via a rapid exchange method.

FIG. 2a is an elevation view, partially in cross section, depicting a delivery system for a medical device with a sheath having a longitudinal joint extending from a proximal end of the sheath to a position proximal a distal end of the sheath.

FIG. 2b is an elevation view, partially in cross section, depicting the delivery system of FIG. 2a with a sheath having a longitudinal joint extending the entire length of the sheath.

FIG. 2c is an elevation view, partially in cross section, of the delivery system of FIG. 2a with a medical device deployed within a vessel of a biological body.

FIG. 3a is a cross section taken from line 3a-3a in FIG. 2a depicting the sheath of the delivery system including the longitudinal joint with other portions of the delivery system removed to more clearly show the sheath.

FIG. 3b is a cross-section view of the longitudinal joint of the present invention depicting mating halves of the longitudinal joint as separated.

FIG. 3c is a cross-sectional view of the longitudinal joint of the present invention depicting another configuration of the mating halves of the longitudinal joint as separated.

FIG. 3d is a cross-sectional view depicting the sheath as manufactured by extrusion.

FIG. 3e is a cross-section view of the longitudinal joint of the present invention depicting the longitudinal joint as a slit having a “s”-shape.

FIG. 3f is a cross-section view of the longitudinal joint of the present invention depicting the sheath having varying thicknesses with the area of the longitudinal joint having a smaller cross section than the surrounding areas.

FIG. 4a is an elevation view depicting an alternative embodiment of the sheath of the delivery device having two lumens.

FIG. 4b is a cross sectional view of the dual-lumen sheath of FIG. 4a taken along line 4b-4b in FIG. 4a.

FIG. 5a is a cross-sectional view depicting a sheath removal ring having a plow.

FIG. 5b is an end view of a sheath removal ring.

FIG. 6 is a cross sectional view depicting a sheath removal ring having a blade and a guide mandrel.

FIG. 7 is a partial perspective view depicting the delivery system of FIG. 2a with the sheath being removed from a biological body and being peeled from a guide wire.

FIG. 8 is an elevation view depicting the sheath of FIG. 7 completely removed from the biological body.

FIG. 9a is a partial perspective view of an alternative embodiment of the sheath of FIG. 2a depicting a dual-lumen sheath with a groove along a length of the sheath and a guide wire extending from one lumen.

FIG. 9b is a partial perspective view depicting the sheath of FIG. 9a being peeled from a guide wire with the guide wire tearing through the groove.

FIG. 9c is a partial perspective view depicting the sheath of FIG. 9a having a slit along the length of the groove and the sheath being peeled from a guide wire with the guide wire exiting through the slit.

### DETAILED DESCRIPTION OF THE PREFERRED EMBODIMENTS

The present invention is directed to a delivery system that can deliver medical devices, such as interventional devices, through the use of an “over-the-wire platform,” and allows the device to be removed through the use of a “rapid exchange” or monorail platform. In use, a medical device is delivered with a guide wire to the intended corporeal vessel site via the over-the-wire mode. After deployment of the device, the delivery sheath is removed from the guide wire, while a single operator holds the guide wire in position. The present invention is particularly useful with fixed wire interventional devices, such as embolic protection systems or filtering devices, wherein the ability to maintain the position of the device within the vessel with minimal movement during sheath removal is required.

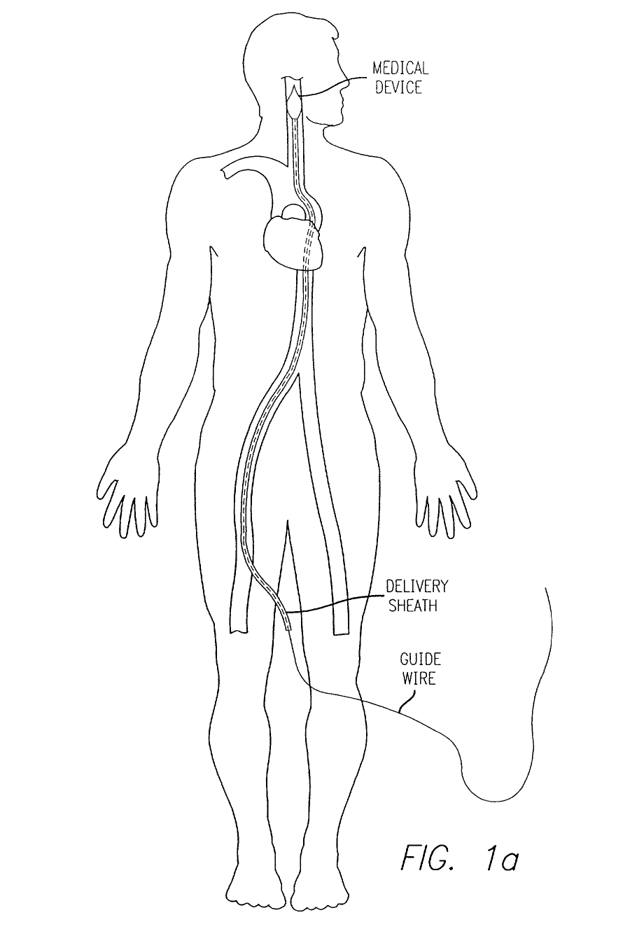

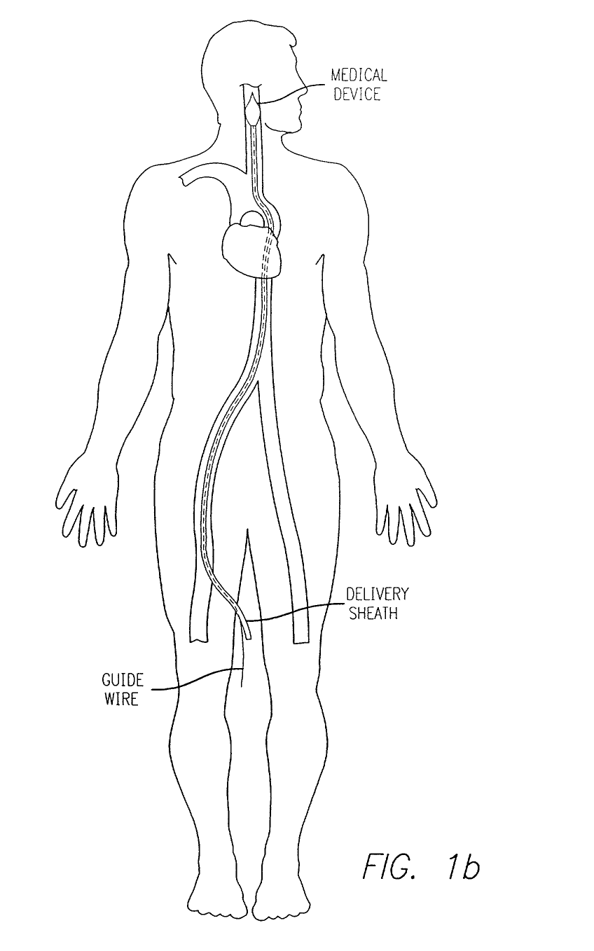

FIG. 1a depicts a prior art catheter for delivering medical devices via the over-the-wire method. The guide wire is more than twice the length of the delivery sheath with more than half the length external to the patient during the delivery of the medical device. The extra length of the guide wire allows the operator to grasp a portion of the guide wire during all stages of delivery sheath removal, but may also require a second person to assist the operator during sheath removal. Coordinating the movements of two people during an interventional procedure can be very difficult and may lead to excessive device movement and vessel trauma or damage. FIG. 1b depicts a rapid exchange or monorail delivery platform for delivering medical devices. The rapid exchange delivery platform allows a much shorter length of guide wire to be external to the patient, permitting the sheath to be removed by only one person. This allows a single operator the ability to maintain the position of a fixed wire medical device within the patient while removing the delivery sheath. This reduces the likelihood of vessel damage or other injury to the patient during an interventional procedure. The present invention combines the benefits associated with each of these approaches. More particularly, the present invention provides for a delivery system having the guide wire housed within the sheath while being guided through the body vessels while the length of the guide wire external the biological body is much shorter than the guide wire for an over-the-wire platform, thereby permitting the sheath retraction to be performed by only one person.

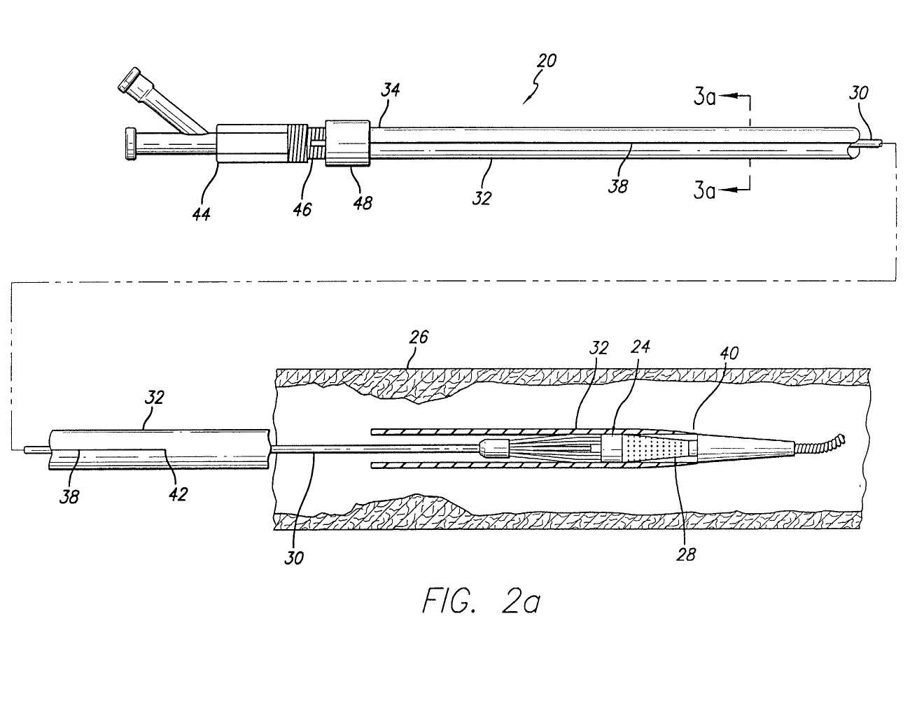

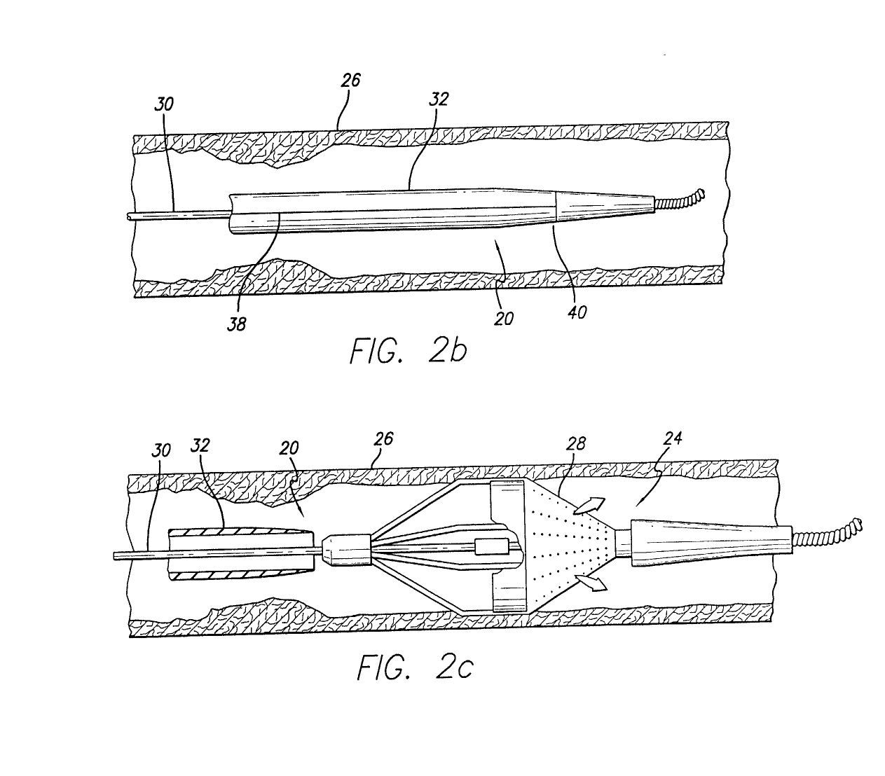

Referring to FIG. 2a, a delivery system 20 incorporating features of the present invention for an embolic protection device 24 is illustrated. The depicted embolic protection device 24 is designed to capture embolic debris that may be created and released into a vessel 26 in a biological body during an interventional procedure. The embolic protection device 24 includes an expandable filter assembly 28 that is coupled to the distal end of an elongate shaft, such as a guide wire 30. A sheath 32 extends coaxially along the guide wire 30 to maintain the expandable filter 28 in its collapsed position until it is ready to be deployed within the vessel 26 in the biological body. The expandable filter 28 is deployed into the vessel 26 in the biological body by retracting the sheath 32 proximally to expose the expandable filter assembly. The expandable filter assembly 28 thus becomes uncovered and immediately begins to expand within the body vessel (see FIG. 2c). It should be appreciated that the embolic protection device depicted herein is just one example of numerous different medical devices that can be utilized in accordance with the present invention, such as balloons and stents. Also, the embodiments of the system and method are illustrated and described herein by way of example only and not by way of limitation.

A longitudinal joint 38 is depicted on the sheath 32 running from a proximal end 34 of the sheath to a location proximal to the distal end 40 of the sheath. The longitudinal joint 38 may be either resealable, or include a longitudinal slit that is non-resealable. In one embodiment, the distal end 42 of the longitudinal joint 38 is about 20 to 40 cm proximal to the distal end 40 of the sheath 32 (FIG. 2a). In another embodiment, the resealable longitudinal joint 38 extends to the distal end 40 of the sheath 32 (see FIG. 2b). The proximal end 34 of the sheath 32 is depicted coupled to a distal portion of a handle, such as a rotatable hemostatic valve 44, via a device, such as a split male Luer lock fitting 46 or other devices well known in the art. The rotatable hemostatic valve 44 allows the guide wire 30 to be placed within an internal lumen (not shown) of the Luer lock fitting 46 while preventing backflow of blood therethrough. A sheath removal ring 48 is depicted distally adjacent the Luer lock fitting 46. The purpose of the longitudinal joint 38 is to facilitate separation of the sheath 32 during retraction of the sheath after deploying the embolic protection device 24, or other medical device, within the body vessel 26. As such, the sheath is permitted to be peeled from the guide wire 30 as the sheath is retracted from the biological body and the guide wire and embolic protection device are retained within the biological body. As will be discussed in more detail below, the longitudinal joint 38 permits the embolic protection device 24 or other medical device to be delivered via an over-the-wire platform and to be removed via a rapid exchange delivery platform.

In one embodiment, the entire length of the guide wire 30 is preferably made from a resiliently deformable material, such as Nitinol. Such material will reduce the likelihood of damage, such as by kinking, to the guide wire 30 during removal of the sheath 32. In another embodiment, only a proximal portion of the guide wire 30 is made from a resiliently deformable material.

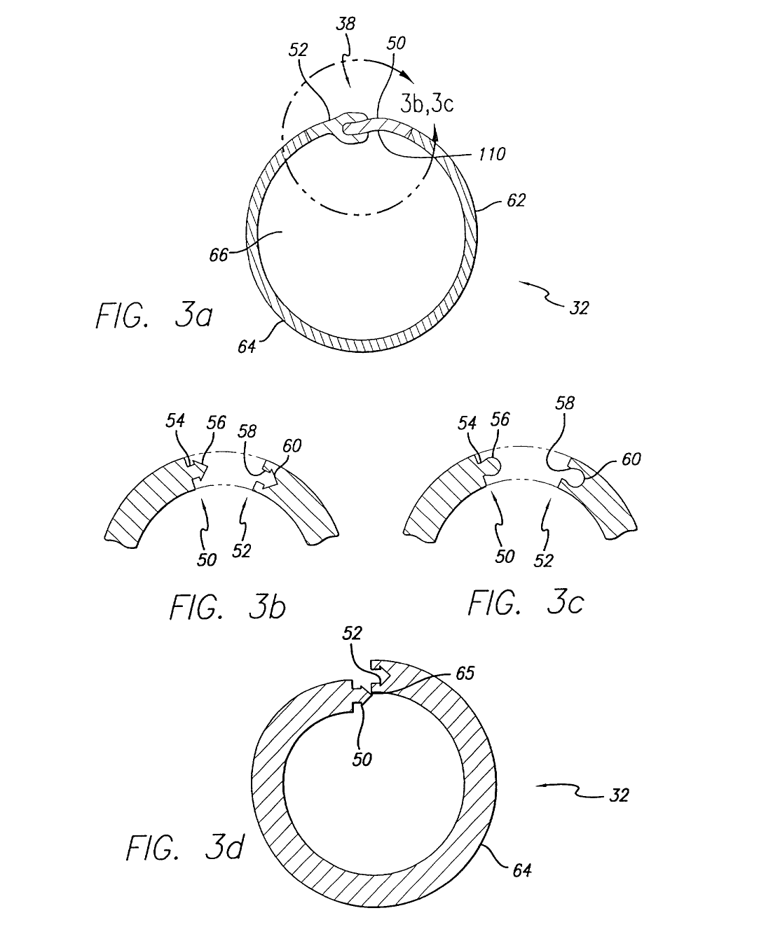

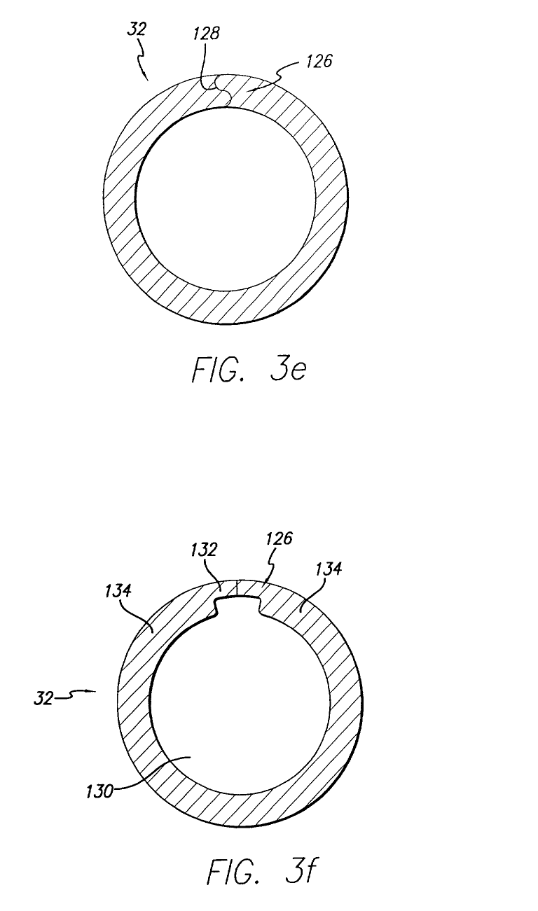

Referring to FIG. 3a, a cross-section of the sheath 32 through the longitudinal joint 38 region depicts the joint having a male portion 50 and a female portion 52 engaged together in a sealing fashion. FIG. 3b depicts the male portion 50 as having a neck-like protrusion 54 that expands to a head 56 of a larger dimension with an arrowhead type shape. The female portion 52 embodies an opening 58 that is at least as large as the neck-like protrusion 54 on the male portion 50, but smaller than the head 56 on the male portion. The opening 58 expands to a larger cavity 60 that can range in size from slightly smaller than the head 56 on the male portion to larger than the head on the male portion. The sheath 32 is made of a flexible, intermediate-durometer polymer such as polyether block amide, known commercially as Pebax™. In one embodiment, the sheath 32 is made of a 72D (Shore “D” hardness scale) hardness scale Pebax™ tube. The flexibility of the sheath 32 material permits the head 56 of the male portion 50 of the longitudinal joint 38 to be forced into and become engaged with the female portion 52. In FIG. 3c, the head 56 on the male portion 50 and the larger cavity 60 of the female portion 52 have a round shape. It should be appreciated that other shapes can be used and that the configurations of the joints 38 depicted in FIGS. 3b and 3c are illustrated and described herein by way of example only and not by way of limitation.

Referring to FIGS. 3e and 3f, a cross-section of the sheath 32 through the longitudinal joint 38 region depicts the joint having a longitudinal slit 126. The longitudinal slit 126 is configured such that the guide wire 30 does not inadvertently dislodge itself from the sheath 32 through the longitudinal slit. One technique to accomplish this is to configure the slit 126 so that it has a curved profile 128, such as an “s”-shape (FIG. 3e). Another technique includes configuring the sheath 32 with varying thicknesses about the lumen 130 such that the area 132 adjacent the slit 126 has a smaller cross-section than the surrounding areas 134 (FIG. 3f). With this configuration, the thicker surrounding areas 134 provide structural support to the thinner area 132 of the slit.

The sheath 32 can be manufactured through the use of many techniques that are well known to those skilled in the art. For instance, the sheath 32 can be manufactured as an extruded member 64 having the shape of an incomplete tube having two open edges defining the longitudinal joint. Extrusions of the male portion 50 and female portion 52 of the longitudinal joint are then coupled to the open edges by methods that are well known in the art, such as by bonding or melding. Alternatively, the sheath 32 can be manufactured with the male portion 50 and female portion 52 in place, such as by extrusion. FIG. 3d depicts one embodiment of the sheath 32 as extruded and prior to engagement of the male portion 50 with the female portion 52. In this embodiment, the tip of the male portion 50 is coupled to an outside edge of a wall defining an opening to the female portion 52 by a membrane 65 that adds support to the sheath during the extrusion process. After the extrusion procedure, the tips of the male 50 and female 52 portions are separated, such as by breaking the membrane, and then engaged. Another method to manufacture the sheath is to extrude the complete sheath as a tube first, followed by a step wherein the joint is added, such as by cutting with a blade or by any other mean known in the art.

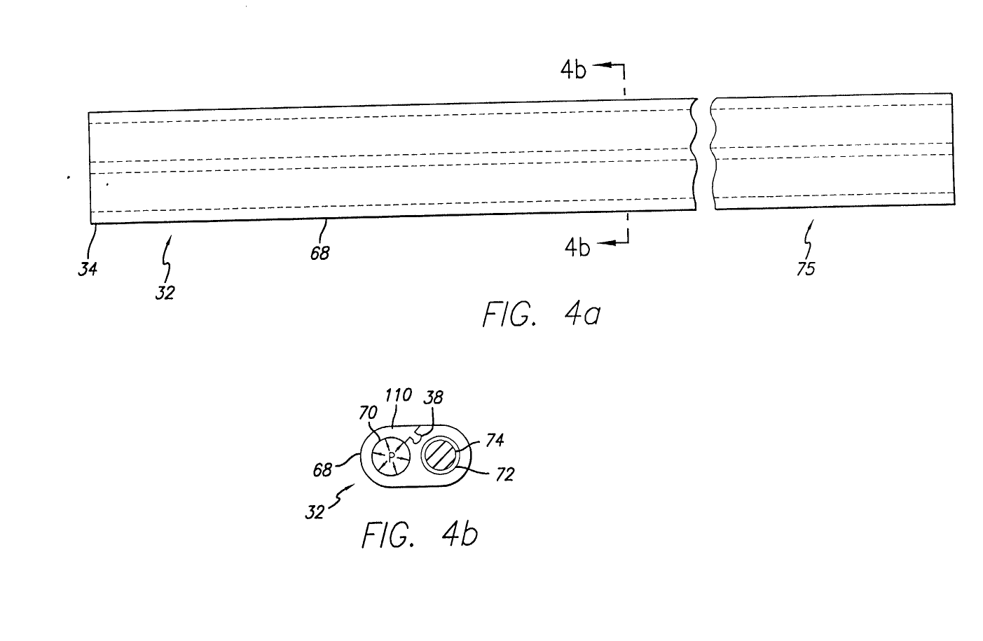

The sheath 32, 62 depicted in FIGS. 2a and 3a embodies a single lumen 66. However, the invention as described may also be used with a multiple-lumen sheath. For instance, FIGS. 4a and 4b depict a dual-lumen sheath 68. The first lumen 70 houses the guide wire (not shown in FIG. 4a or 4b) while the second lumen 72 houses a support mandrel 74. The support mandrel 74 increases the columnar strength of the delivery system 20. The support mandrel 74 extends from the proximal end 34 of the sheath 32 to a distal portion 75 of the sheath. The longitudinal joint 38 is connected to the first lumen 70 to facilitate removal of the dual-lumen sheath 62 from the guide wire 30 as the dual-lumen sheath is retracted from the biological body.

Prior to insertion into the biological body, the lumens 66, 70, 72 (FIGS. 3a and 4b) of the sheath are flushed with a biologically compatible liquid, such as a saline solution, in order to purge air from the lumens that may otherwise escape into the biological body. During flushing, the lumens 66, 70,72 are subjected to pressures up to 8 atm. Therefore, when the male portion 50 and female portion 52 of the longitudinal joint 38 are engaged, the first lumen 70 of FIG. 4b, or the single lumen 66 of FIG. 3a, is capable of holding pressure up to and over 8 atm.

When flushing a sheath 32 having the longitudinal slit 126, an end of the sheath is flushed with the biologically compatible liquid, with the liquid exiting through the opposite end of the sheath. A secondary packaging tube (not shown) that extends over and encapsulates the longitudinal slit 126 section may be used during the flushing process to ensure that air is purged from the sheath. The packaging tube is removed from the sheath 32 before insertion of the sheath into the patient.

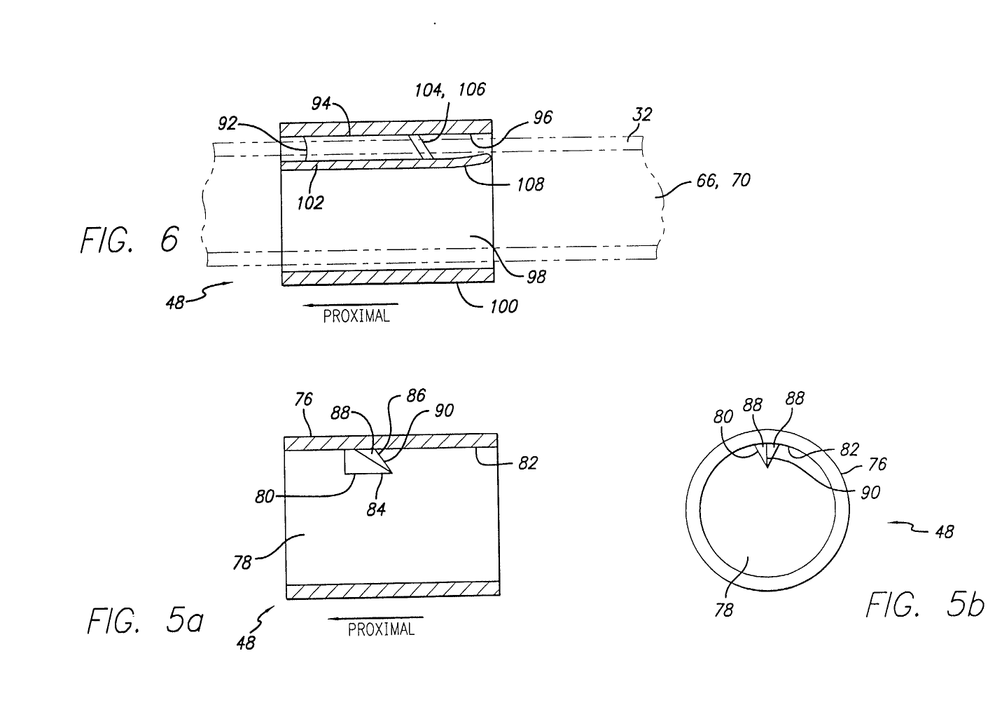

Referring to FIGS. 5a and 5b, the sheath removal ring 48 includes a ring 76 having a lumen or internal bore 78 that is shaped to match the outside surface of the sheath 32 (FIG. 2a). The lumen 78 is large enough to fit over the sheath 32 and to permit the longitudinal joint 38 to separate during relative movement between the sheath and the removal ring 48. A plow 80 is coupled to a surface 82 of the lumen 78. The plow 80 is oriented longitudinally and, as shown in FIG. 5b, has a generally triangular cross section that is long enough to extend through a wall defining the sheath 32 and into the lumen 66, 70 of the sheath 32 (FIGS. 3a and 4b). In one embodiment, the plow 80 is positioned to align with the longitudinal joint 38 of the sheath 32 (FIG. 2a). FIG. 5a depicts that the distal end 84 of the plow 80 has an angled surface 86 that extends distally as the surface extends away from the lumen surface 82. Two symmetrical facets 88 forming moldboards are also on the distal side of the plow. The facets 88 come together at a point at the distal end 84 of the plow 80 and extend proximally and toward the lumen surface 82 with the intersection 90 of the facets forming a plowshare. During use, the sheath removal ring 48 is located around the proximal end 34 of the sheath 32 (FIG. 2a), and distal relative movement of the sheath removal ring 48 over the sheath 32 causes the distal end 84 of the plow 80 to penetrate and separate the longitudinal joint 38 of the sheath 32.

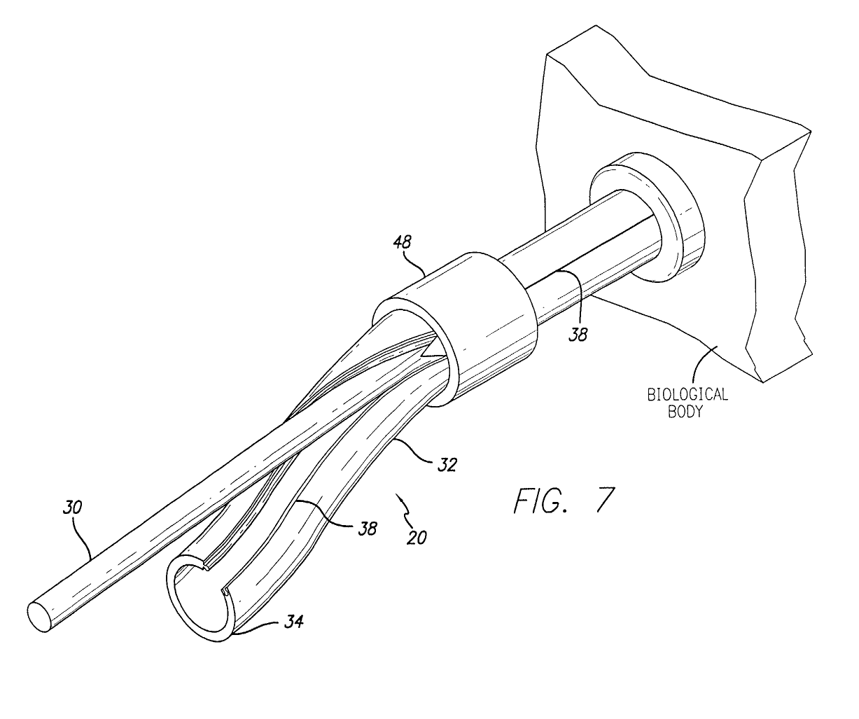

Referring to FIG. 6, another embodiment of the sheath removal ring 48 replaces the plow with a blade 92. A bottom edge 94 of the blade 92 is coupled to the surface 96 of the lumen 98 of the ring 100. The blade 92 is oriented longitudinally with respect to the ring 100. The top edge 102 of the blade 92 is dimensioned to extend into the lumen 66, 70 of the sheath 32 (FIGS. 3a and 4b). The distal end 104 of the blade 92 has a cutting edge 106 that angles distally as it extends away from the lumen surface 96 of the ring 100. A guide mandrel 108 that runs longitudinally through the lumen 98 of the ring 100 is coupled to the top edge 102 of the blade 92, which places the guide mandrel within the lumen 66,70 of the sheath 32 (FIGS. 3a and 4b). During use, the sheath removal ring 48 is located around the proximal end 34 of the sheath 32 (FIG. 2a), and distal relative movement of the ring over the sheath causes the distal cutting edge 106 of the blade 92 to slice through a wall 110 of the sheath 32 (FIGS. 3a and 4b), such as through the longitudinal joint 38. The guide mandrel 108 ensures that the wall 110 of the sheath 32 does not deflect inwardly away from the blade 92 during cutting and also reduces the likelihood of the blade causing damage to other components within the lumen 66, 70 of the sheath 32.

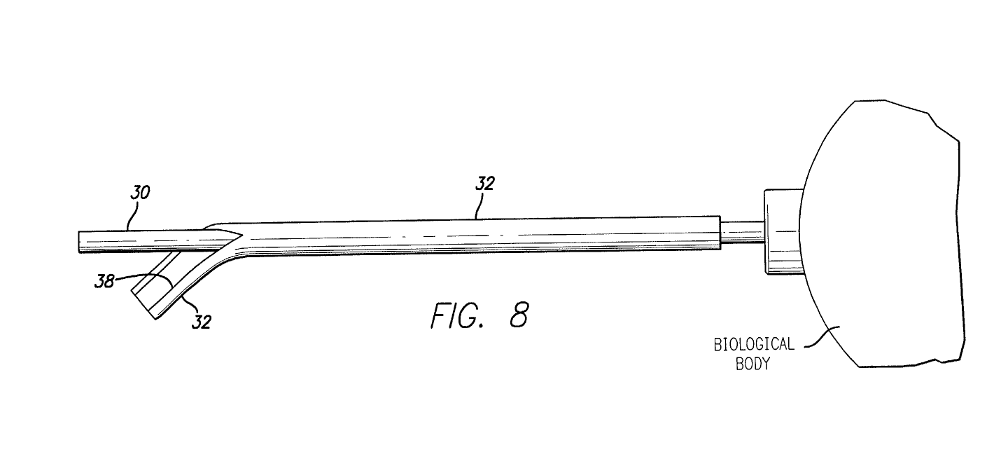

Referring again to FIGS. 2a and 2c, the embolic protection device 24 or other medical device is delivered via the delivery system 20 to a site within a vessel 26 of the biological body. The operator deploys the depicted embolic protection device 24 by retracting the sheath 32 proximally with one hand while maintaining the longitudinal placement of the guide wire 30 with the other hand to prevent the embolic protection device 24 from moving. The sheath 32 is then removed from the biological body so that other devices, such as interventional devices, can be routed along the guide wire 30 to the site in the vessel 26. The sheath 32 is removed by first removing the handle, or rotatable hemostatic valve 44, and Luer lock fitting 46, or other device coupling the distal portion of the handle to the proximal portion of the sheath, from the proximal end 34 of the sheath 32. Then, while using one hand to maintain the longitudinal placement of the sheath 32 and guide wire 30, the other hand translates the sheath removal ring 48 distally along the sheath toward the biological body, thereby causing the plow 80 or blade 92 to separate the longitudinal joint 38. The operator then uses one hand to peel the portion of the sheath 32 proximal the sheath removal ring 48 from the guide wire 30 via the opened longitudinal joint 38 while using the other hand to maintain the longitudinal placement of the guide wire (FIG. 7). While one hand simultaneously maintains the longitudinal placement of the guide wire 30 and restrains the sheath removal ring 48, the other hand removes the remainder of the sheath 32 from the biological body by simultaneously pulling the sheath proximally through the sheath removal ring and peeling the sheath from the guide wire. When the portion of the sheath 32 distal the longitudinal joint 38 is retracted from the biological body (FIG. 8), the sheath removal ring 48 and the remainder of the sheath can be removed from the guide wire 30 with one hand while the other hand maintains the longitudinal placement of the guide wire. The guide wire 30 is then free so that other devices can be routed along the guide wire to the site in the vessel 26.

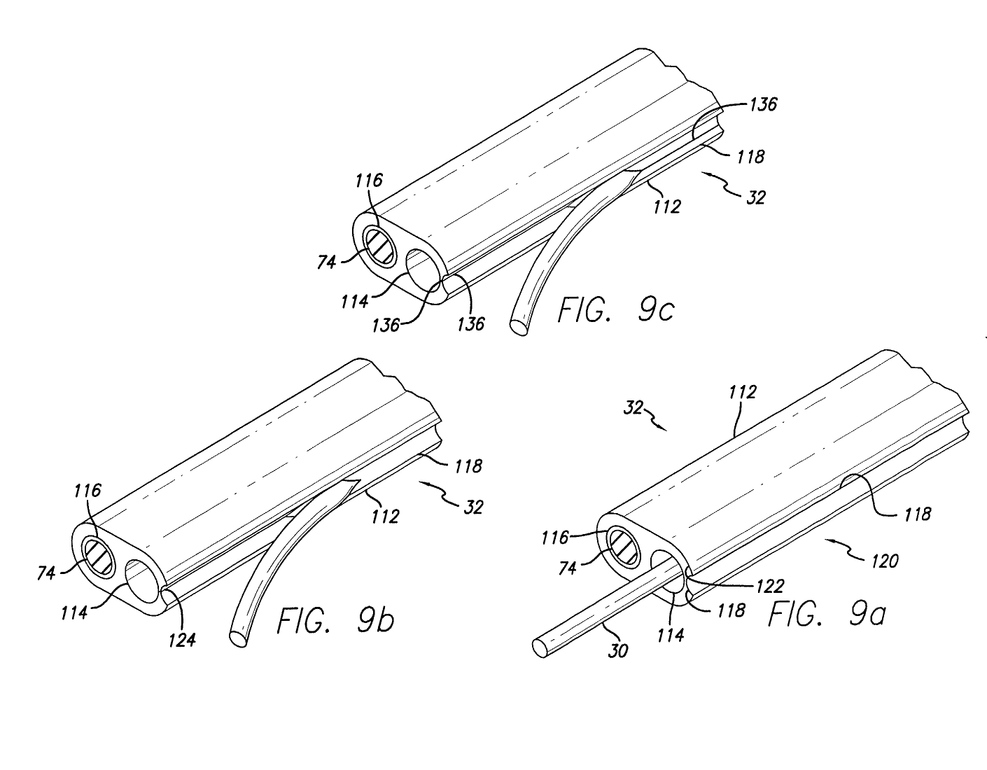

FIG. 9a depicts another embodiment of the sheath 32 that is similar to the dual lumen sheath 68 that is shown in FIGS. 4a and 4b. Like the sheath 68 of FIGS. 4a and 4b, this embodiment includes a dual-lumen sheath 112 having a first lumen 114 for housing the guide wire 30 and a second lumen 116 for housing the support mandrel 74. However, the sheath 112 of FIG. 9a does not include the longitudinal joint 38. Rather, the sheath 112 has a groove 118 that runs longitudinally along the side 120 of the sheath and throughout the length of the sheath. The groove 118 reduces the thickness of the wall 122 of the first lumen 114. Alternatively, the groove 118 can be replaced with a reduced wall thickness at any location around the edge of the first lumen. Regardless of whether a groove 118 or a reduced wall thickness is used, the lumens 114, 116 are capable of being pressurized up to and over 8 atm. to accommodate flushing of the lumens to purge air from the lumens.

Referring to FIG. 9b, the groove 118, or reduced wall thickness, eliminates the need for the sheath removal ring 48. Instead, after the guide wire 30 and embolic protection device 24 are deployed, the handle and the device coupling the handle to the sheath are removed and the sheath 112 is peeled from the guide wire while maintaining the longitudinal placement of the guide wire by causing the guide wire to tear through the groove 118 or reduced wall, thereby forming a longitudinal joint 124 therethrough. After being peeled from the guide wire 30, the newly formed longitudinal joint 124 closes up but does not become sealed. Alternatively, as depicted in FIG. 9c, the sheath 112 can include a slit 136 extending from the proximal end of the delivery sheath to a location approximately 20 to 40 centimeters proximal to the distal end of the sheath. In this embodiment, the sheath 112 is peeled from the guide wire 30 by causing the guide wire to pull through the slit 136.

At the completion of the operational procedure, the sheath 32 can be used in its assembled form as a retrieval device to recover the guide wire 30 and embolic protection device 24 or other medical device. To retrieve the guide wire 30 and embolic protection device 24, the distal end 40 of the sheath 32 is routed along the proximal end of the guide wire while maintaining the longitudinal placement of the guide wire. The sheath 32 is routed distally along the guide wire 30 as the guide wire is inserted into the lumen 66, 70, 114 of the sheath 32 through the longitudinal joint 38, 124 or slit 126, 136 until the entire lumen houses the guide wire. When the distal end 40 of the sheath 32 reaches the embolic protection device 24, the guide wire 30 is held in place while the sheath is translated distally over the embolic protection device, thereby collapsing the embolic protection device and restraining it within the lumen 66, 70, 114. The sheath 32, guide wire 30 and embolic protection device 24 may then be retracted from the vessel 26 of the biological body simultaneously.

In view of the foregoing, it is apparent that the systems of the present invention substantially enhance the efficiency of deploying and recovering embolic protection devices or other medical devices, such as balloon catheters or stent delivery systems. Further modifications and improvements may additionally be made to the system and method disclosed herein without departing from the scope of the present invention. Accordingly, it is not intended that the invention be limited, except as by the appended claims.

What is claimed is:

1. A delivery sheath for a medical device, the delivery sheath comprising:
an elongate tube having a proximal end and a distal end; and
a resealable longitudinal joint.

2. The delivery sheath of claim 1, the longitudinal joint further comprising:
a first side and a second side;
the first side including a protrusion having a neck leading to a head, the head being larger than the neck; and
the second side including an opening leading to a cavity, the opening being smaller than the head of the first side and at least as large as the neck of the first side, and the cavity being within a range of slightly smaller to larger than the head of the first side.

3. The sheath of claim 1, further comprising:
a single lumen.

4. The sheath of claim 3, wherein:
the longitudinal joint extends throughout an entire length of the sheath.

5. The sheath of claim 3, wherein:
the longitudinal joint extends from a proximal end of the sheath to a position proximal a distal end of the sheath.

6. The sheath of claim 5, wherein:
the longitudinal joint extends distally to a position between 20 and 40 centimeters proximal a distal end of the sheath.

7. The sheath of claim 3, wherein:
the longitudinal joint has a depth extending from an external surface of the sheath to a surface of the lumen.

8. The sheath of claim 3, wherein:
the lumen is capable of being pressurized up to and over 8 atm.

9. The sheath of claim 1, further comprising:
at least a first lumen and a second lumen.

10. The sheath of claim 9, wherein:
the longitudinal joint extends an entire length of the sheath.

11. The sheath of claim 9, wherein:
the longitudinal joint extends from a proximal end of the sheath to a position proximal a distal end of the sheath.

12. The sheath of claim 11, wherein:
the longitudinal joint extends distally to a position between 20 and 40 centimeters proximal the distal end of the sheath.

13. The sheath of claim 9, wherein:
the longitudinal joint has a depth extending from an external surface of the sheath to a surface of the first lumen.

14. The sheath of claim 9, wherein:
the first lumen is capable of being pressurized up to and over 8 atm.

15. A delivery sheath for a medical device, the delivery sheath comprising:
an elongate tube having a proximal end and a distal end; and
a longitudinal joint.

16. The delivery sheath of claim 15, the longitudinal joint comprising:
a contoured profile having a s-shape.

17. The delivery sheath of claim 15, the longitudinal joint comprising:
a slit located within a section that is thinner than its surrounding areas.

18. The sheath of claim 15, further comprising:
a single lumen.

19. The sheath of claim 18, wherein:
the longitudinal joint extends throughout an entire length of the sheath.

20. The sheath of claim 18, wherein:
the longitudinal joint extends from a proximal end of the sheath to a position proximal a distal end of the sheath.

21. The sheath of claim 18, wherein:
the longitudinal joint has a depth extending from an external surface of the sheath to a surface of the lumen.

22. The sheath of claim 15, further comprising:
at least a first lumen and a second lumen.

23. The sheath of claim 22, wherein:
the longitudinal joint has a depth extending from an external surface of the sheath to a surface of the first lumen.

24. A delivery sheath for an embolic protection device, the delivery sheath comprising:
an elongate tube having at least a first lumen and a second lumen; and
a reduced cross sectional area positioned adjacent the first lumen.

25. The sheath of claim 24, wherein:
the reduced cross sectional area is defined by a groove penetrating an outer surface of the sheath.

26. The sheath of claim 24, wherein:
the first lumen is capable of being pressurized up to and over 8 atm.

27. The sheath of claim 24, further comprising:
a longitudinal joint located between the reduced cross-sectional area and the first lumen and extending therebetween.

28. The sheath of claim 27, wherein:
the longitudinal joint extending from a proximal end of the sheath to a position between 20 and 40 centimeters proximal a distal end of the sheath.

29. A delivery system for delivering medical devices, the delivery system comprising:
a sheath including an elongate tube having at least one lumen extending a length of the sheath and a resealable longitudinal joint;
a guide wire distributed throughout the length of the at least one lumen;
a handle disposed at a proximal portion of the sheath;
a device coupling the proximal portion of the sheath to a distal portion of the handle; and
a device configured to split the longitudinal joint and to allow the sheath to be removed from the guide wire.

30. The delivery system of claim 29, the splitting device further comprising:
a ring having a lumen, the ring fitting over the sheath; and
a plow coupled to a surface defined by the lumen of the ring, a height of the plow being sufficient to extend into the lumen of the sheath and including a distal portion configured to enter and split the longitudinal joint during relative longitudinal movement between the ring and the sheath.

31. The delivery system of claim 29, the splitting device further comprising:
a ring having a lumen, the ring fitting over the sheath; and
a blade aligned with a longitudinal axis of the ring and being coupled to the surface of the lumen of the ring, a height of the blade being sufficient to extend into the lumen of the sheath and including a distal edge for cutting the sheath during relative longitudinal movement between the ring and the sheath.

32. The delivery system of claim 31, the ring further comprising:
a guide mandrel coupled to the edge of the blade, the guide mandrel configured to be positioned within the lumen of the sheath.

33. The delivery system of claim 29, wherein:
the device coupling the proximal portion of the sheath to the distal portion of the handle is split along the length of the device.

34. A delivery system for delivering medical devices, the delivery system comprising:
a sheath including an elongate tube having at least one lumen extending a length of the sheath longitudinal joint;
a guide wire distributed throughout the length of the at least one lumen;
a handle disposed at a proximal portion of the sheath; and
a device coupling the proximal portion of the sheath to a distal portion of the handle;
a device configured to allow the sheath to split and be removed from the guide wire.

35. The delivery system of claim 34, wherein:
the device coupling the proximal portion of the sheath to the distal portion of the handle is split along the length of the device.

36. A method for deploying a medical device within a biological body vessel via a delivery system, the delivery system including a sheath having a resealable longitudinal joint, a guide wire distributed throughout a length of the sheath, a handle disposed at a proximal portion of the sheath, a device coupling the proximal portion of the sheath to a distal portion of the handle, and a sheath removal ring, comprising:
introducing the medical device with the delivery system into the body vessel;
advancing the medical device to the desired location within the body vessel;
deploying the medical device at the desired location within the body vessel; and
removing the sheath from the guide wire by causing the sheath removal ring to split the sheath along the resealable longitudinal joint.

37. The method of claim 36, deploying the medical device further comprising:
holding the guide wire in a relatively steady position while extracting the sheath proximally relative to the guide wire to expose the medical device.

38. The method of claim 36, removing the sheath further comprising:
detaching the handle from the proximal portion of the sheath;
advancing the sheath removal ring distally while holding the guide wire and the sheath in a relatively steady position, thereby splitting the longitudinal joint at the proximal portion of the sheath;
pulling the sheath proximally while holding the sheath removal ring and the guide wire in a relatively steady position until a distal end of the longitudinal joint is external the biological body;
removing the sheath removal ring while holding the guide wire in a relatively steady position; and
removing the remainder of the sheath from the guide wire while holding the guide wire in a relatively steady position.

39. The method of claim 36, removing the sheath further comprising:
removing the device coupling the proximal portion of the sheath to the distal portion of the handle.

40. A method for deploying a medical device within a biological body vessel via a delivery system, the delivery system including a sheath having a longitudinal joint, a guide wire distributed throughout a length of the sheath, a handle disposed at a proximal portion of the sheath, a device coupling the proximal portion of the sheath to a distal portion of the handle, and a sheath removal ring, comprising:
introducing the medical device with the delivery system into the body vessel;
advancing the medical device to the desired location within the body vessel;
deploying the medical device at the desired location within the body vessel; and
removing the sheath from the guide wire by causing the sheath removal ring to split the sheath along the longitudinal joint.

41. The method of claim 40, deploying the medical device further comprising:
holding the guide wire in a relatively steady position while extracting the sheath proximally relative to the guide wire to expose the medical device.

42. The method of claim 40, removing the sheath further comprising:
detaching the handle from the proximal portion of the sheath;
advancing the sheath removal ring distally while holding the guide wire and the sheath in a relatively steady position, thereby splitting the longitudinal joint at the proximal portion of the sheath;
pulling the sheath proximally while holding the sheath removal ring and the guide wire in a relatively steady position until a distal end of the longitudinal joint is external the biological body;
removing the sheath removal ring while holding the guide wire in a relatively steady position; and
removing the remainder of the sheath from the guide wire while holding the guide wire in a relatively steady position.

43. The method of claim 40, removing the sheath further comprising:
removing the device coupling the proximal portion of the sheath to the distal portion of the handle.

44. A method for deploying a medical device within a biological body vessel via a delivery system, the delivery system including a sheath having at least a first lumen and a second lumen and a reduced cross sectional area positioned adjacent the first lumen, a guide wire distributed throughout a length of the sheath, a handle disposed at a proximal portion of the sheath, and a device coupling the proximal portion of the sheath to a distal portion of the handle:
introducing the medical device with the delivery system into the body vessel;
advancing the medical device to the desired location within the body vessel;
deploying the medical device at the desired location within the body vessel; and
removing the sheath from the guide wire by causing the guide wire to tear through the reduced cross sectional area along the length of the sheath.

45. The method of claim 44, deploying the medical device further comprising:
holding the guide wire in a relatively steady position while extracting the sheath proximally relative to the guide wire to expose the medical device.

46. The method of claim 44, removing the sheath further comprising:
detaching the handle from the proximal portion of the sheath;
pulling the sheath proximally while holding the guide wire in a relatively steady position until a distal end of the groove is external the biological body; and
removing the remainder of the sheath from the guide wire while holding the guide wire in a relatively steady position.

47. The method of claim 44, removing the sheath further comprising:
removing the device coupling the proximal portion of the sheath to the distal portion of the handle.

48. A method of manufacturing a sheath having a resealable longitudinal joint comprising a male portion and a female portion, comprising:
extruding a tubular-shape body having the male portion disengaged from the female portion, a tip of the male portion and an outside edge of the female portion being coupled by a membrane;
breaking the membrane to separate the tips of the male portion and the female portion; and
engaging the male portion with the female portion.
